(19) United States
(12) Patent Application Publication
(10) Pub. No.: US 2020/0297563 A1
(43) Pub. Date: Sep. 24, 2020

(54) ADJUSTING METHOD AND ADJUSTING SYSTEM FOR HUMAN BODY SUPPORT DEVICE

(71) Applicant: EBIO TECHNOLOGY INC., New Taipei City (TW)

(72) Inventor: DER-CHIH SHR, Taoyuan City (TW)

(21) Appl. No.: 16/823,057

(22) Filed: Mar. 18, 2020

Related U.S. Application Data
(60) Provisional application No. 62/821,404, filed on Mar. 20, 2019.

(51) Int. Cl. A61G 7/057; A47C 31/12; A47C 27/08

(52) U.S. Cl. CPC A61G 7/05769; A47C 31/123; A61B 5/1116; A61G 2203/10; A47C 27/083

(57) ABSTRACT

An adjusting method and an adjusting system are provided. The adjusting method for human body support device includes the following steps: extracting a pressure distribution information when a user is lying on a human body support device; analyzing a posture of the user based on the pressure distribution information; analyzing a human musculoskeletal distribution information of the skeleton-muscle of the user and a plurality of coordinate values corresponding to the skeleton-muscle of the user based on the pressure distribution information; obtaining a plurality of high-risk regions corresponding to the plurality of pressure regions based on the human musculoskeletal distribution information, a plurality of coordinate values corresponding to the skeleton-muscle of the user, and the posture of the user; and adjusting a pressure value configuration of an airbag module of the human body support device based on the plurality of high-risk regions.

### CROSS-REFERENCE TO RELATED PATENT APPLICATION

This application claims priority from the U.S. Provisional Patent Application Ser. No. 62/821,404 filed Mar. 20, 2019, which application is incorporated herein by reference in its entirety.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to an adjusting method and an adjusting system for a human body support device, and more particularly to an adjusting method and an adjusting system for a human body support device based on the structure of a human body.

### BACKGROUND OF THE DISCLOSURE

The hardness of a traditional mattress device cannot be adjusted for users who spend long hours lying down. Since the pressure against various parts of the user's body is not adjusted, pressure ulcers can commonly occur when the user lies on the mattress device for long hours at a time.

Therefore, it is an important subject in the industry to provide an adjusting method and an adjusting system for a human body support device that can adjust the human body support device based on the body structure of different users.

### SUMMARY OF THE DISCLOSURE

In response to the above-referenced technical inadequacies, the present disclosure provides an adjusting method for human body support device. The adjusting method for human body support device includes steps: extracting a pressure distribution information when a user lies upon a human body support device; analyzing a posture of the user based on the pressure distribution information; analyzing a human musculoskeletal distribution information of a skeleton-muscle of the user and a plurality of coordinate values corresponding to the skeleton-muscle of the user based on the pressure distribution information; obtaining a plurality of high-risk regions corresponding to the plurality of pressure regions based on the human musculoskeletal distribution information, a plurality of coordinate values corresponding to the skeleton-muscle of the user, and the posture of the user; and adjusting a pressure value configuration of an airbag module of the human body support device based on the plurality of high-risk regions.

In one aspect, the present disclosure provides an adjusting system for human body support device. The adjusting system for human body support device includes a control device, and a human body support device. The human body support device includes a pressure detecting module, a processing module, an airbag module, a pressure adjusting module, a communication module, and a power module. The human body support device is in communication with the control device. The pressure detecting module is disposed on a side of the human body support device to detect a pressure distribution information when a user lies on the human body support device. The processing module is electrically connected to the pressure detecting module. The airbag module being electrically connected to the processing module. The pressure adjusting module is electrically connected to the processing module. The pressure adjusting module is connected to the airbag module. The airbag module performs pressure adjustment. The communication module is electrically connected to the processing module. The human body support device is in communication with the control device through the communication module. The power module is electrically connected to the processing module, the pressure adjusting module, the pressure detecting module, and the communication module. The processing module provides the pressure distribution information to the control device. The control device analyzes a posture of the user based on the pressure distribution information. The control device analyzes a human musculoskeletal distribution information of a skeleton-muscle of the user and a plurality of coordinate values corresponding to the skeleton-muscle of the user based on the pressure distribution information. The control device obtains a plurality of high-risk regions corresponding to the plurality of pressure regions based on the musculoskeletal distribution information, the plurality of coordinate values corresponding to the skeleton-muscle of the user, and the posture of the user. The control device provides a pressure control signal to the pressure adjusting module of the human body support device based on the plurality of high-risk regions to adjust a pressure value configuration of the airbag module.

Therefore, the adjusting method and adjusting system for human body support device provided by the present disclosure perform pressure adjustment based on the weak part of the force-bearing part of a human body. The adjusting method and adjusting system for human body support device not only adjust the data in the pressure distribution information, but also adjust the pressure values of the airbag module based on each of user body, such that the user would feel more comfortable and the user's experience can be effectively improved.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a flowchart of an adjusting method for human body support device of a first embodiment of the present disclosure.

FIG. 2 is a flowchart of machine learning used in the adjusting method for human body support device of the first embodiment of the present disclosure.

FIG. 3 is a schematic view of an adjusting system for human body support device of the first embodiment of the present disclosure.

FIG. 4 is a schematic view of a pressure distribution information of a user lying on a human body support device.

FIG. 5 is a schematic view of a user lying on the human body support device of the first embodiment of the present disclosure.

FIG. 6 is another schematic view of a user lying on the human body support device of the first embodiment of the present disclosure.

FIG. 7 is a schematic view of a human body support device before a pressure configuration value of the present disclosure is adjusted.

FIG. 8 is a schematic view of the human body support device after the pressure configuration value of the present disclosure is adjusted.

FIG. 9 is a block diagram of an adjusting system for human body support device of a second embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

### First Embodiment

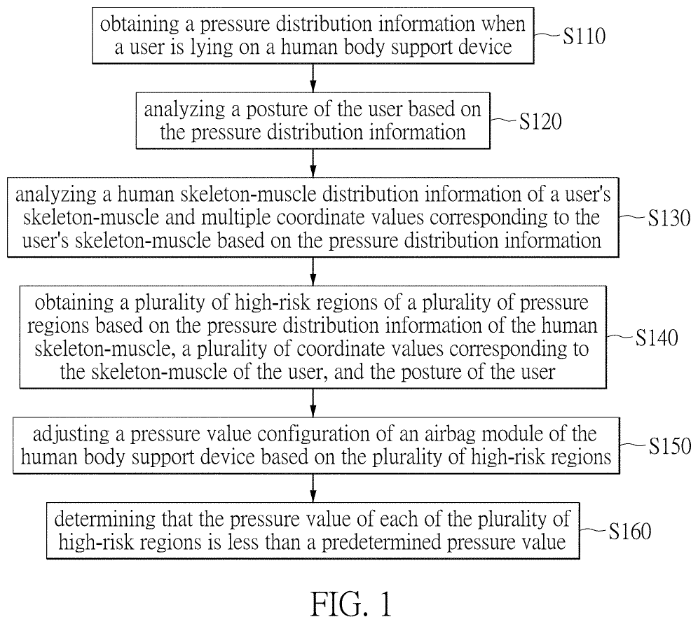

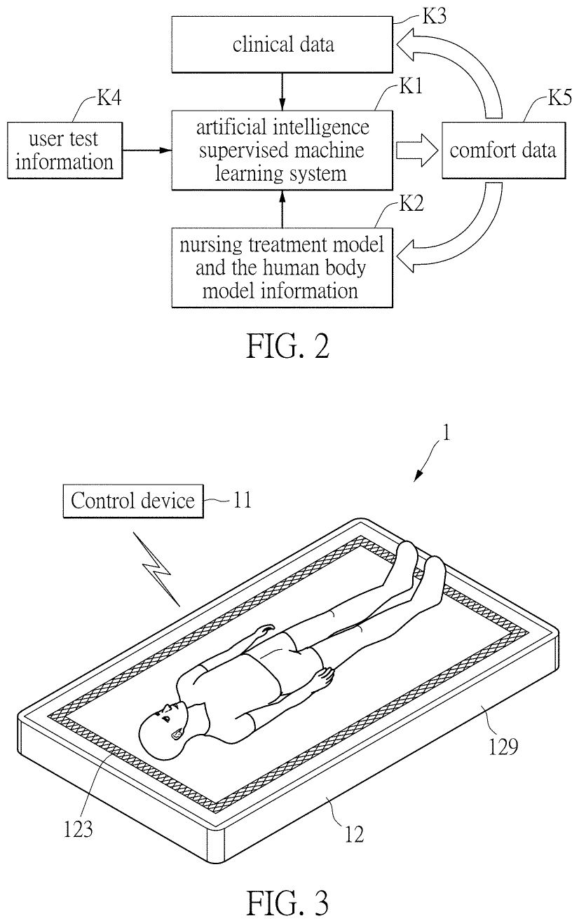

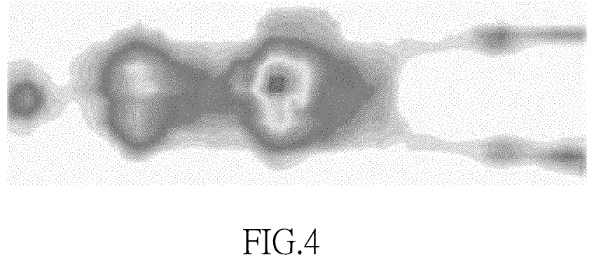

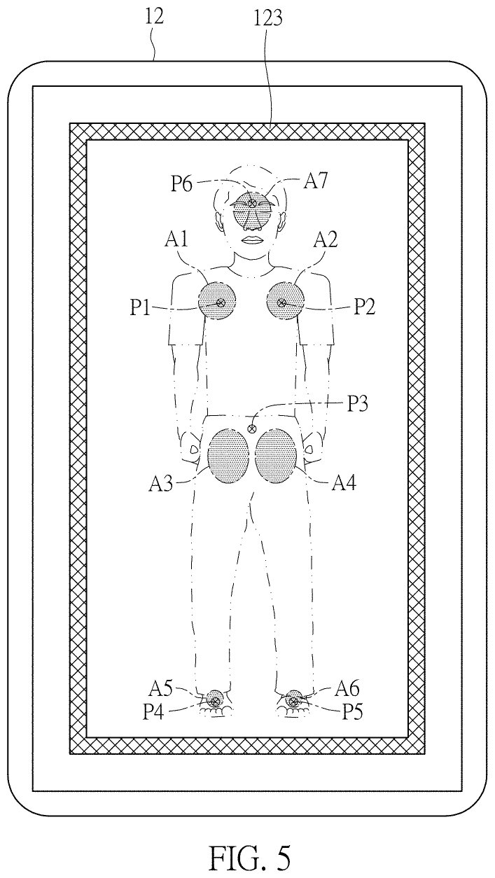

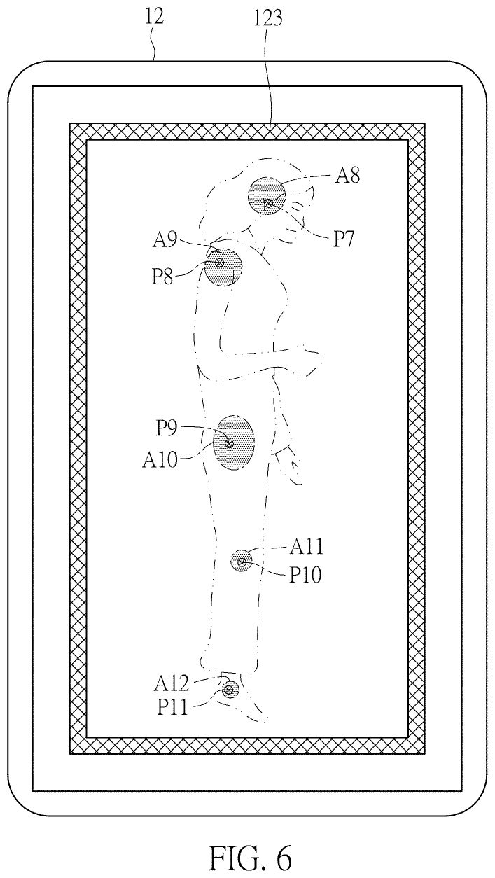

Referring to FIG. 1, FIG. 2, FIG. 3, FIG. 4, FIG. 5, and FIG. 6, FIG. 1 is a flowchart of an adjusting method for human body support device of a first embodiment of the present disclosure. FIG. 2 is a flowchart of machine learning used in the adjusting method for human body support device of the first embodiment of the present disclosure. FIG. 3 is a schematic view of an adjusting system for human body support device of the first embodiment of the present disclosure. FIG. 4 is a schematic view of a pressure distribution information of a user lying on a human body support device. FIG. 5 is a schematic view of a user lying on the human body support device of the first embodiment of the present disclosure. FIG. 6 is another schematic view of a user lying on the human body support device of the first embodiment of the present disclosure.

A method for adjusting a human body support device of this embodiment is adapted for an adjusting system for a human body support device 1. The adjusting system for human body support device 1 includes a control device 11 and a human body support device 12.

The human body support device 12 includes a processing module 120, an airbag module 121, a pressure adjusting module 122, a pressure detecting module 123, a communication module 124, a power module 125, and a device body 129.

The adjusting method for a human body support device of this embodiment includes following steps:

extracting a pressure distribution information when the user is lying on a human body support device (step S110);

analyzing a posture of the user based on the pressure distribution information (step S120);

analyzing a human musculoskeletal distribution information of the user's skeleton-muscle and multiple coordinate values corresponding to the user's skeleton-muscle based on the pressure distribution information (step S130);

obtaining a plurality of high-risk regions corresponding to a plurality of pressure regions based on the pressure distribution information of the human skeleton-muscle, a plurality of coordinate values corresponding to the skeleton-muscle of the user, and the posture of the user (step S140);

adjusting a pressure configuration state of an airbag module of the human body support device based on the plurality of high-risk regions (step S150); and

determining that the pressure value of each of the plurality of high-risk regions is less than a predetermined pressure value (step S160).

In step S110, the human body support device 12 is a mattress device. In other embodiments, the human body support device 12 is a seat cushion device or a cushion device. The pressure detecting module 123 of the human body support device 12 is a flexible pressure detecting module and is disposed on a side of the human body support device 12. The pressure detecting module 123 includes a plurality of pressure detecting units (not shown). The pressure detecting units (not shown) are arranged in a matrix on a flexible substrate (not shown), such as a plastic substrate or a cloth substrate, and the present disclosure is not limited thereto. In the embodiment, a plurality of pressure detecting units (not shown) of the pressure detecting module 123 may be wholly or partially disposed on the upper surface of the support device 12. In addition, a plurality of pressure detecting units (not shown) of the pressure detecting module 123 should be disposed in at least a user's back area and a user's hip area. In other words, a plurality of pressure detecting units (not shown) of the pressure detecting module 123 may be respectively disposed in the back area and the hip area of the user. In addition, a plurality of pressure detecting units (not shown) of the pressure detecting module 123 may be disposed in a back area, a waist region, and a hip area, namely, a torso region of the user.

When the user lies on the human body support device 12, the pressure detecting module 123 detects the pressure distribution information applied by the user of the human body support device 12. The pressure distribution information of the user is transmitted to the processing module 120.

In step S120, the processing module 120 transmits the pressure distribution information of the user to a control device 11 through the communication module 124. The control device 11 analyzes a posture of the user based on the pressure distribution information of the user. The pressure distribution information is at least a two-dimensional pressure distribution image. The control device 11 determine the posture of the user when the user lies on the human body support device 12 based on multiple characteristic pressure points in the pressure distribution information, such as those of the head, shoulders, scapula, elbow, wrist, hip, knee, and ankle.

In the embodiment, the postures of the user lying on the human body support device 12 include a supine posture, a prone posture, and a side-lying posture (including a left-side lying posture and a right-side lying posture). The posture of the user is mainly a posture of the user's upper body, and other irregular postures are included in the above-mentioned lying postures. In other embodiments, types of the posture can be adjusted and designed based on actual requirements, which is not limited in the present disclosure. When the user is lying on the support device 12 in a supine position, and the hip area is taken as an example, the pressure distribution information of the hip area has 2-3 pressure regions with pressure peak value. However, when the user is lying on the support device 12 in a side-lying posture, since the human body is resting on the support device 12 with the hip in the side-lying position, the pressure distribution information of the hip area has a single pressure region with peak value, and the pressure peak value of the side-lying posture is generally higher than that of the supine posture.

The control device 11 can determine the posture of the user based on several determining factors: (1) a body pressure profile parameter PA1; and (2) a local peak point distribution parameter PA2 of pressure region. The body pressure profile parameter PA1 includes a coverage region of the user's back area and the hip area, and an appearance pressure distribution characteristic. The local peak point distribution parameter PA2 of pressure region is a number of pressure peaks in different pressure regions as previously described. The control device 11 may perform weight distribution based on the above two factors, for example, a first weighting factor W1 times the body pressure profile parameter PA1, and a second weighting factor W2 times the local peak point distribution parameter PA2 of pressure region. Therefore, the control device 11 combines the determining factors and the weighting factors to perform machine learning.

In the embodiment, the control device 11 can determine and perform machine learning process based on the following formula 1:

LF=PA1*W1+PA2*W2− formula 1.

LF represents a user's lying posture parameter. The control device 11 can adjust the first weighting factor W1 and the second weighting factor W2 through lying postures of a plurality of users to improve on an accuracy of determining the lying postures of the users. In the embodiment, when the user's lying posture parameter LF is greater than a posture reference value, the lying posture is determined to be a supine posture. When the user's lying posture parameter LF is less than the posture reference value, it is determined to be a side-lying posture. In addition, in this embodiment, the first weighting factor W1, the second weighting factor W2, the body pressure profile parameter PA1, and the local peak point distribution parameter PA2 of pressure region can also be corrected by confirming whether the posture of the user is successfully determined to be the supine posture or the side-lying posture.

In other embodiments, the human body support device 12 is a seat cushion device or a cushion device. In other words, the human body support device 12 may only carry a part of the human body and a part of the weight of the user. Through different ways of installation of the human body support device 12, such as horizontal, vertical, or inclined placement, and the difference in the amount of force applied by the user based on different positions, the control device 11 can determine the posture of the user lying on the human support device 12.

The control device 11 is a desktop computer, a notebook computer, a local server, a remote server, a mobile phone, a tablet computer, or a wearable electronic device.

In step S130 and step S140, the control device 11 analyzes the skeleton-muscle correlation and distribution state of the user based on the pressure distribution information analysis to obtain the human musculoskeletal distribution information and multiple coordinate values corresponding to the user's skeleton-muscle. Furthermore, the control device 11 also obtains a plurality of high-risk regions corresponding to a plurality of pressure regions based on the pressure distribution information of the human skeleton-muscle, a plurality of coordinate values corresponding to the skeleton-muscle of the user, and a posture of the user. In addition, the control device 11 also extracts the respective maximum pressure points of the multiple pressure regions A1-A12 (as shown in FIG. 5 and FIG. 6) in the pressure distribution information as a reference.

In step S130, the control device 11 may estimate a height value and a weight value of the user based on the pressure distribution information of the user to calculate the distribution information of the human skeleton-muscle of the user. In addition, the control device 11 can obtain the height value and weight value of the user to calculate the musculoskeletal distribution information of the user. The musculoskeletal distribution information can show a distribution of human bones and muscles, and weak regions of the skeleton-muscle structure.

Therefore, the control device 11 can obtain a plurality of high-risk regions P1-P11 corresponding to a plurality of pressure regions based on the human musculoskeletal distribution information, multiple coordinate values corresponding to the skeleton-muscles of the user and the posture of the user (as shown in FIG. 5 and FIG. 6).

In addition, the control device 11 can determine a current force-bearing state of the user based on the user's human musculoskeletal distribution information and the maximum pressure value of each of the pressure regions A1-A12 (as shown in FIG. 5 and FIG. 6) in the pressure distribution information.

Furthermore, the control device 11 obtains the human musculoskeletal distribution information based on the human musculoskeletal distribution information of the user and a posture obtained through the pressure distribution information. Therefore, the control device 11 further compares the human musculoskeletal distribution information and the pressure distribution information based on the user's posture. In this way, multiple high-risk regions P1-P11 in the pressure distribution information can be obtained (as shown in FIG. 5 and FIG. 6).

In the above-mentioned comparison process, the position with the maximum pressure value in each pressure region is used to determine whether the position with the maximum pressure value in each pressure region is a weak region of the human body. If the position with the maximum pressure value in each pressure region is a weak region, the position with the maximum pressure value in each pressure region is marked as a high-risk region. If the position with the maximum pressure value in each pressure region is not a weak region, the weak regions around the maximum pressure value of each pressure region are marked as high-risk regions.

Since each of the pressure regions with the highest pressure value is generally not the weakest region of the human structure, the position where the bones protrude from in each pressure region is often weaker. Therefore, the high-risk region is not the same as the maximum pressure in each of the pressure regions.

In step S150, the control device 11 transmits a control signal to the processing module 120 of the human body support device 12 based on the multiple high-risk regions, such that the processing module 120 adjusts a pressure value configuration of the airbag module 121. In the embodiment, the airbag module 121 includes a plurality of airbag units (not shown), and the airbag units (not shown) are arranged in a matrix in the human body support device 12. Moreover, the pressure value of each of the airbag units (not shown) can be independently adjusted. Therefore, the control signal transmitted by the control device 11 can accurately control each of the airbag units (not shown) of the airbag module 121.

Referring to FIG. 2, FIG. 2 is a flowchart of machine learning used in the adjusting method for the human body support of the present disclosure. An artificial intelligence supervised machine learning system K1 receives the nursing treatment model and the human body model information provided by a human factor model K2, the real-time human body information provided by a user test information K4, and a clinical data provided by a clinical data K3 to generate a comfort data K5.

Since the artificial intelligence supervised machine learning system K1 in this case continuously learns based on known data, the comfort data K5 of the human comfort effect verification will be fed back to the nursing treatment model and the human factor model K2 and clinical data K3 to update the human treatment information of the nursing treatment model and the human factor model K2, and the clinical data of the clinical data K3.

In other words, the human body support device 12 can adjust the pressure value of the airbag module 121 to adjust the pressure distribution information of the user lying on the human body support device 12, such that the pressure value in the high-risk region can be reduced for improving on the discomfort and risks caused by lying down, such as pressure ulcer or poor blood circulation. In the embodiment, the pressure value of each high-risk region will be reduced by 20%, and is adjusted based on the user's pressure distribution information.

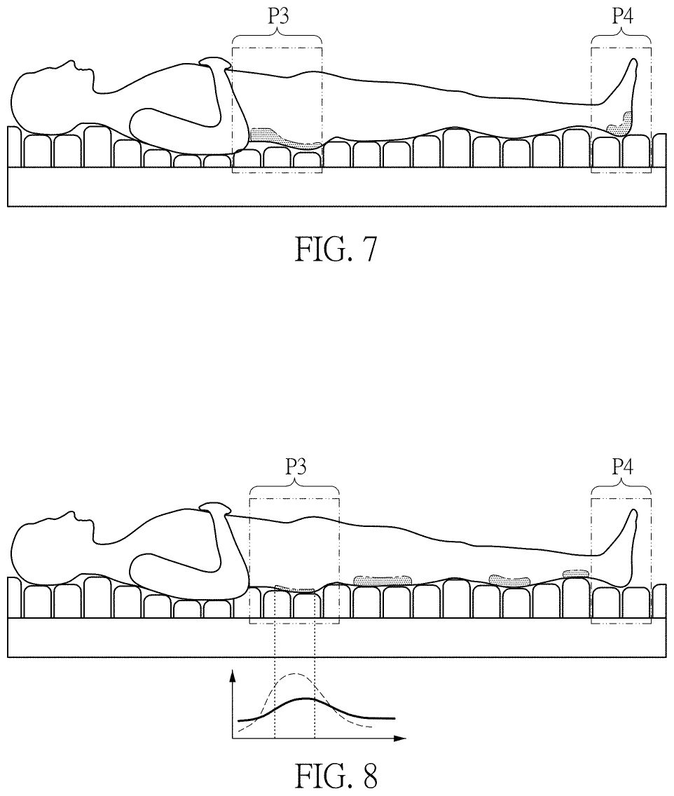

Referring to FIG. 7 and FIG. 8, FIG. 7 is a schematic view of a human body support device before a pressure configuration value of the present disclosure is adjusted. FIG. 8 is a schematic view of the human body support device after the pressure configuration value of the present disclosure is adjusted.

In step S160, the control device 11 reconfirms the pressure distribution information of the user after adjusting the airbag module 121 to confirm that the user can be supported comfortably and with low risk. In other words, the control device 11 confirms whether the respective pressure value of each of the high-risk regions P1-P11 is less than a predetermined pressure value. In the embodiment, the predetermined pressure values of each high-risk region are different, and are determined based on the human musculoskeletal distribution information of the user.

In other words, the adjusting system for the human body support device of this embodiment detects the pressure distribution information of the user, and detects a lying posture or reclining posture of the user based on the pressure distribution information. In addition, the human body support device of the embodiment also calculates the correlation of the skeleton-muscle distribution of the user and related coordinate value based on the pressure distribution information. Furthermore, the multiple high-risk regions are obtained based on the user's posture, the skeleton-muscle distribution correlation, and related coordinate values, and the pressure distribution of the user is readjusted. In other words, the pressure in the high-risk region is adjusted to improve on pressure ulcers caused by poor blood circulation.

Referring to FIG. 7 and FIG. 8, FIG. 7 is a schematic view of a human body support device before a pressure configuration value of the present disclosure is adjusted. FIG. 8 is a schematic view of the human body support device after the pressure configuration value of the present disclosure is adjusted.

In FIG. 7 and FIG. 8, the pressure of the airbag module corresponding to the high-risk regions P3 and P4 is adjusted to readjust the pressure distribution of the user. In FIG. 8, the pressure distribution in the high-risk region P3 is reduced due to the redistribution of the pressure of the airbag module. Therefore, the pressure condition of the high-risk region P3 is readjusted, such that a pressure concentration of the pressure distribution information is shifted.

### Second Embodiment

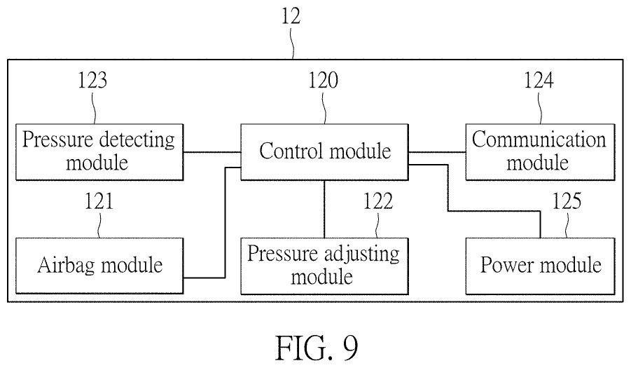

Referring to FIG. 9, FIG. 9 is a block diagram of an adjusting system for human body support device of a second embodiment of the present disclosure.

An adjusting system for human body support device 1 includes a control device 11 and a human body support device 12. The control device 11 is in communication with the human body support device 12.

The human body support device 12 includes a processing module 120, an airbag module 121, a pressure adjusting module 122, a pressure detecting module 123, a communication module 124, a power module 125, and a device body 129. The processing module 120 is electrically connected to the pressure adjusting module 122, the pressure detecting module 123, the communication module 124, and the power module 125.

The pressure adjusting module 122 is connected to the pressure detecting module 123. In the embodiment, the pressure adjusting module 122 includes a pump (not shown), multiple inflation valves (not shown), and multiple relief valves (not shown). The pump (not shown) of the pressure adjusting module 122 connected to the airbag module 121 by a plurality of inflation valves (not shown) and a plurality of relief valves (not shown), and at least one hollow inflation air pipe (not shown). The airbag module 121 includes a plurality of airbag units (not shown). A plurality of airbag units (not shown) of the airbag module 121 are arranged in a matrix in the device body 129.

The processing module 120 transmits a pressure control signal to the pump (not shown) of the pressure adjusting module 122 to inflate or deflate the airbag module 121. In the embodiment, multiple airbag units (not shown) of the airbag module 121 can be inflated or deflated independently.

The pressure detecting module 123 is disposed on a side of the human body support device 12. In the embodiment, a plurality of pressure detecting units (not shown) of the pressure detecting module 123 may be wholly or partially disposed on an upper surface of the support device 12. In addition, a plurality of pressure detecting units (not shown) of the pressure detecting module 123 should be disposed at least in regions corresponding to a back area and a buttock region of the user. In other words, a plurality of pressure detecting units (not shown) of the pressure detecting module 123 may be respectively disposed in the back area and the hip area of the user. In addition, a plurality of pressure detecting units (not shown) of the pressure detecting module 123 may be disposed in regions corresponding to a back area, a waist region, and a hip area of the user, namely, a torso region of the user.

The communication module 124 is electrically connected to the processing module 120. The human body support device 12 is in communication with the control device 11 through the communication module 124. The communication module 124 is a wired communication module, a Wi-Fi communication module, a Bluetooth® communication module, a LoRa communication module, a Sigfox communication module, or an NB-IoT communication module.

The power module 125 is electrically connected to the processing module 120, the pressure adjusting module 122, the pressure detecting module 123, and the communication module 124. The power module 125 is configured for supplying power to the processing module 120, the pressure adjusting module 122, the pressure detecting module 123, and the communication module 124. In the embodiment, the power module 125 is a DC-to-DC voltage converter or an AC-to-DC voltage converter.

Firstly, when the user is lying on the human body support device 12, the pressure detecting module 123 detects the pressure distribution information applied by the user of the human body support device 12. The pressure distribution information of the user is transmitted to the processing module 120.

The processing module 120 provides the pressure distribution information to the control device 11 through the communication module 124. The control device 11 analyzes a posture of the user based on the pressure distribution information.

The pressure distribution information is at least a two-dimensional pressure distribution image. The control device 11 determines a posture of the user lying on the human support device 12 based on multiple characteristic pressure points in the pressure distribution information, such as the head, shoulders, and scapula, elbows, wrists, hip, knees, ankles, etc.

In the embodiment, the posture of the user lying on the human body support device 12 include a supine posture, a prone posture, and a side-lying posture (including a left-side lying posture and a right-side lying posture). The posture of the user is mainly a posture of the user's upper body, and other irregular postures are included in the above-mentioned lying postures. In other embodiments, types of the posture can be adjusted and designed based on actual requirements, which is not limited in the present disclosure. When the user is lying on the support device 12 in a supine position, and the hip area is taken as an example, the pressure distribution information of the hip area has 2-3 pressure regions with pressure peak value. However, when the user is lying on the support device 12 in a side-lying posture, since the human body is resting on the support device 12 with the hip in the side-lying position, the pressure distribution information of the hip area has a single pressure region with peak value, and the pressure peak value of the side-lying posture is generally higher than that of supine posture.

The control device 11 can determine the posture of the user based on several determining factors: (1) a body pressure profile parameter PA1; and (2) a local peak point distribution parameter PA2 of pressure region. The body pressure profile parameter PA1 includes a coverage region of the user's back area and the hip area, and an appearance pressure distribution characteristic. The local peak point distribution parameter PA2 of pressure region is a number of pressure peaks in different pressure regions as previously described. The control device 11 may perform weight distribution based on the above two factors, for example, a first weighting factor W1 times the body pressure profile parameter PA1, and a second weighting factor W2 times the local peak point distribution parameter PA2 of pressure region. Therefore, the control device 11 combines the determining factors and the weighting factors to perform machine learning.

In the embodiment, the control device 11 can determine and perform machine learning process based on the following formula 2:

LF=PA1*W1+PA2*W2− formula 2.

LF represents a user's lying posture parameter. The control device 11 can adjust the first weighting factor W1 and the second weighting factor W2 by a plurality of posture of users to improve on an accuracy of determining the lying posture of the users. In the embodiment, when the user's lying posture parameter LF is greater than a posture reference value, it is determined to be a supine posture. When the user's lying posture parameter LF is less than the posture reference value, it is determined to be a side-lying posture. In addition, in this embodiment, the first weighting factor W1, the second weighting factor W2, the body pressure profile parameter PA1, and the local peak point distribution parameter PA2 of pressure region can also be corrected by confirming whether the posture of the user is successfully determined to be the supine posture or the side-lying posture.

In other embodiments, the human body support device 12 is a seat cushion device or a cushion device. In other words, the human body support device 12 may only carry a part of the human body and a part of the weight of the user. Due to different installation ways of the human body support device 12, such as horizontal, vertical, or inclined placement, and the difference in the amount of force applied by the user based on different positions. Therefore, the control device 11 can determine the posture of the user lying on the human support device 12.

The control device 11 is a desktop computer, a notebook computer, a local server, a remote server, a mobile phone, a tablet computer, or a wearable electronic device.

In step S130 and step S140, the control device 11 analyzes the skeleton-muscle correlation and distribution state of the user based on the pressure distribution information analysis to obtain the human musculoskeletal distribution information and multiple coordinate values corresponding to the user's skeleton-muscle. Furthermore, the control device 11 also obtains a plurality of high-risk regions corresponding to a plurality of pressure regions based on the pressure distribution information of the human skeleton-muscle, a plurality of coordinate values corresponding to the skeleton-muscle of the user, and a posture of the user. In addition, the control device 11 also extracts the respective maximum pressure points of the multiple pressure regions A1-A12 (as shown in FIG. 5 and FIG. 6) in the pressure distribution information as a reference.

The control device 11 may estimate a height value and a weight value of the user based on the pressure distribution information of the user to calculate the distribution information of the human skeleton-muscle of the user. In addition, the control device 11 can obtain the height value and weight value of the user to calculate the musculoskeletal distribution information of the user. The musculoskeletal distribution information can show a distribution of human bones and muscles, and weak regions of the skeleton-muscle structure.

Therefore, the control device 11 can obtain a plurality of high-risk regions P1-P11 corresponding to a plurality of pressure regions based on the human musculoskeletal distribution information, multiple coordinate values corresponding to the skeleton-muscles of the user and the posture of the user.

In addition, the control device 11 can determine a current force state of the user based on the user's human musculoskeletal distribution information and the maximum pressure value of each pressure region A1-A12 (as shown in FIG. 5 and FIG. 6) in the pressure distribution information.

Furthermore, the control device 11 obtains the human musculoskeletal distribution information based on the human musculoskeletal distribution information of the user and a posture obtained through the pressure distribution information. Therefore, the control device 11 further compares the human musculoskeletal distribution information and pressure distribution information based on the user's posture. In this way, multiple high-risk regions P1-P11 in the pressure distribution information can be obtained.

In the above-mentioned comparison process, the position of the maximum pressure value in each pressure region is used to determine whether or not the position of the maximum pressure value in each pressure region is a weak region of the human body. If the position of the maximum pressure value in each pressure region is a weak region, the position of the maximum pressure value in each pressure region is marked as a high-risk region. If the position of the maximum pressure value in each pressure region is not a weak region, the weak regions around the maximum pressure value of each pressure region are marked as high-risk regions.

Since each of the pressure regions with the highest pressure value is generally not the weakest region of the human structure, the structure of a bony prominence in each pressure region is often weaker. Therefore, the high-risk region is not the same as the maximum pressure in each of the pressure regions.

The control device 11 transmits a control signal to the processing module 120 of the human body support device 12 based on the multiple high-risk regions, such that the processing module 120 adjusts a pressure value configuration of the airbag module 121. In the embodiment, the airbag module 121 includes a plurality of airbag units (not shown), and the airbag units (not shown) are arranged in a matrix in the human body support device 12. Moreover, the pressure of each of the airbag units (not shown) can be independently adjusted. Therefore, the control signal transmitted by the control device 11 can accurately control each of the airbag units (not shown) of the airbag module 121.

The human body support device 12 can adjust the pressure value of the airbag module 121 to adjust the pressure distribution information of the user lying on the human body support device 12, such that the pressure value in the high-risk region can be reduced to reduce discomfort and risks caused by lying, such as pressure ulcers or poor blood circulation. In the embodiment, the pressure value of each high-risk region will be reduced by 20%, and adjusted based on the user's pressure distribution information. Furthermore, the control device 11 reconfirms the pressure distribution information of the user after adjusting the airbag module 121 to confirm that the user can be supported comfortably and with low risk. In other words, the control device 11 confirms whether the respective pressure value of each of the high-risk regions P1-P11 is less than a predetermined pressure value. In the embodiment, the predetermined pressure values of each high-risk region are different, and are determined based on the user's human musculoskeletal distribution information.

In conclusion, the adjusting method and adjusting system for human body support device provided by the present disclosure perform pressure adjustment based on the weak part of the force-bearing part of a human body. The adjusting method and adjusting system for human body support device not only adjust the data in the pressure distribution information, but also adjust the pressure values of the airbag module based on each of user body, such that the user would feel more comfortable and the user's experience can be effectively improved.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An adjusting method for human body support device, comprising:
extracting a pressure distribution information when a user lies upon a human body support device;
analyzing a posture of the user based on the pressure distribution information;
analyzing a human musculoskeletal distribution information of a skeleton-muscle of the user and a plurality of coordinate values corresponding to the skeleton-muscle of the user based on the pressure distribution information;
obtaining a plurality of high-risk regions corresponding to the plurality of pressure regions based on the human musculoskeletal distribution information, a plurality of coordinate values corresponding to the skeleton-muscle of the user, and the posture of the user; and
adjusting a pressure value configuration of an airbag module of the human body support device based on the plurality of high-risk regions.

2. The adjusting method of claim 1, further comprising the following step:
confirming that the pressure value of each of the high-risk regions is less than a predetermined pressure value.

3. The adjusting method of claim 1, wherein the human body support device includes a pressure detecting module being disposed on a side of the human body support device, the airbag module including a plurality of airbag units, and the pressure value configuration including pressure values of the plurality of airbag units.

4. The adjusting method of claim 1, wherein, in the step of analyzing a posture of the user, the human body support device determines the posture of the user lying on the human body support device based on a configuration state of the human body support device.

5. The adjusting method of claim 1, wherein the human body support device is a mattress device.

6. The adjusting method of claim 5, wherein the posture at least includes a supine posture, a prone posture, and a side-lying posture.

7. The adjusting method of claim 1, wherein the human body support device is a seat cushion device or a cushion device.

8. An adjusting system for human body support device, comprising:
a control device;
a human body support device being in communication with the control device, the human body support device including:
a pressure detecting module being disposed on a side of the human body support device to detect a pressure distribution information when a user lies on the human body support device;
a processing module being electrically connected to the pressure detecting module;
an airbag module being electrically connected to the processing module;
a pressure adjusting module being electrically connected to the processing module, the pressure adjusting module being connected to the airbag module, and the airbag module performing pressure adjustment;
a communication module being electrically connected to the processing module, and the human body support device being in communication with the control device through the communication module; and
a power module being electrically connected to the processing module, the pressure adjusting module, the pressure detecting module, and the communication module;
wherein the processing module provides the pressure distribution information to the control device, and the control device analyzes a posture of the user based on the pressure distribution information;
wherein the control device analyzes a human musculoskeletal distribution information of a skeleton-muscle of the user and a plurality of coordinate values corresponding to the skeleton-muscle of the user based on the pressure distribution information, and the control device obtains a plurality of high-risk regions corresponding to the plurality of pressure regions based on the musculoskeletal distribution information, the plurality of coordinate values corresponding to the skeleton-muscle of the user, and the posture of the user;
wherein the control device provides a pressure control signal to the pressure adjusting module of the human body support device based on the plurality of high-risk regions to adjust a pressure value configuration of the airbag module.

9. The adjusting system of claim 8, wherein the human body support device is a mattress device, a seat cushion device, or a cushion device.
